(19) United States
(12) Patent Application Publication
(10) Pub. No.: US 2022/0175802 A1
(43) Pub. Date: Jun. 9, 2022

(54) COMBINATION OF MINOCYCLINE AND BENZOYL PEROXIDE AND METHOD OF USE THEREOF

(71) Applicant: Sol-Gel Technologies Ltd., Ness Ziona (IL)

(72) Inventors: Moshe ARKIN, Kfar Shmaryahu (IL); Karine NEIMANN, Ness Ziona (IL); Danil FINKEL-MOISEEV, Rehovot (IL)

(73) Assignee: Sol-Gel Technologies Ltd., NessZiona (IL)

(21) Appl. No.: 17/440,780

(22) PCT Filed: Mar. 19, 2020
(86) PCT No.: PCT/IL2020/050338

§ 371 Date: Sep. 19, 2021

Related U.S. Application Data
(60) Provisional application No. 62/820,544, filed on Mar. 19, 2019.

(51) Int. Cl. A61K 31/65; A61K 31/327; A61K 9/00; A61K 9/06; A61K 47/34; A61K 47/44; A61K 47/10; A61K 47/02; A61K 47/12

(52) U.S. Cl. CPC A61K 31/65; A61K 31/327; A61K 9/0014; A61K 9/06; A61K 47/12; A61K 47/44; A61K 47/10; A61K 47/02; A61K 47/34

(57) ABSTRACT

This invention is directed to a regimen administration of combination of minocycline and benzoyl peroxide and uses thereof for treating a skin disorder such as acne or rosacea.

### FIELD OF THE INVENTION

This invention is directed to a regimen administration of combination of minocycline and benzoyl peroxide and uses thereof for treating a skin disorder such as acne or rosacea.

### BACKGROUND OF THE INVENTION

Tetracyclines are broad-spectrum antibiotic, which are routinely used orally for the treatment of dermatological conditions, such as acne and rosacea. However, despite their high therapeutic value, tetracyclines are very unstable, and they are known to be incompatible with many formulation excipients, including water, various protic substances and oxidizing agents.

Minocycline is one type of tetracycline antibiotics which exhibits strong antibiotic action and is widely used in therapy, primarily to treat acne and rosacea at a once daily dose of 100 mg. However, minocycline is an unstable substance.

Benzoyl Peroxide (BPO) is also known for treating acne. Encapsulated BPO was found for treatment of rosacea by Sol-Gel (U.S. Pat. No. 9,687,465). Benzoyl peroxide acts by destroying P. acnes, the bacteria that causes the condition acne. It acts as an antiseptic and as an oxidizing agent, reducing the number of comedones, or blocked pores. Topical administration of BPO causes severe adverse events such as erythema, irritation, burning, stinging, scaling and itching. To treat mild to moderate acne, benzoyl peroxide is used in combination of antibiotics or topical retinoids. The combination of benzoyl peroxide with either erythromycin or clindamycin is synergistic and well-tolerated. [Dutil, M., Skin Therapy Letters, 2010, 15(10), 5-7; Titus. S., Am Fam Physician. 2012 Oct. 15; 86(8):734-740].

Sol-Gel process has been used to encapsulate various active ingredients, thus isolating the active ingredient from the environment. U.S. Pat. Nos. 6,303,149, 6,238,650, 6,468,509, 6,436,375, US2005037087, US2002064541, and International publication Nos. WO 00/09652, WO 00/72806, WO 01/80823, WO 03/03497, WO 03/039510, WO00/71084, WO05/009604, and WO04/81222, disclose sol-gel microcapsules and methods for their preparation. EP 0934773 and U.S. Pat. No. 6,337,089 teach microcapsules containing core material and a capsule wall made of organopolysiloxane, and their production. EP 0 941 761 and U.S. Pat. No. 6,251,313 also teach the preparation of microcapsules having shell walls of organopolysiloxane. U.S. Pat. No. 4,931,362 describes a method of forming microcapsules or micromatrix bodies having an interior water-immiscible liquid phase containing an active, water-immiscible ingredient. Microcapsules prepared by a sol-gel process are also disclosed in GB2416524, U.S. Pat. No. 6,855,335, WO03/066209.

Acne vulgaris (cystic acne or “acne”) is a common human skin disease, characterized by areas of skin with redness, comedones (blackheads and whiteheads), papules (pinheads), pustules (pimples), nodules (large papules) and possibly scarring. Acne affects mostly skin with the densest population of sebaceous follicles; these areas include the face, the upper part of the chest, and the back. Severe acne is inflammatory, but acne can also manifest in noninflammatory forms. The lesions are caused by changes in pilosebaceous units, skin structures consisting of a hair follicle and its associated sebaceous gland, changes that require androgen stimulation. Acne occurs most commonly during adolescence, and often continues into adulthood.

Rosacea is a chronic disease of inflammatory dermatitis that mainly affects the median part of the face and the eyelids of certain adults. It is characterized by telangiectatic erythema, dryness of the skin, papules and pustules. Conventionally, rosacea develops in adults from the ages of 30 to 50; it more frequently affects women, although the condition is generally more severe in men. Rosacea is a primitively vascular condition whose inflammatory stage lacks the cysts and comedones characteristic of common acne.

There still is a widely recognized need for a treatment for acne and rosacea. Treatment with benzoyl peroxide and antibiotics like minocycline is not obvious, especially due to the sensitivity between the two agents (benzoyl peroxide can oxidize and thereby degrade minocycline).

### SUMMARY OF THE INVENTION

In one aspect, this invention provides a dual chamber dispenser, having a first chamber charged with a first composition comprising from about 2% w/w to about 10% w/w benzoyl peroxide, and a second chamber charged with a second composition comprising from about 0.5% w/w to about 10% w/w of minocycline or a pharmaceutically acceptable salt thereof, wherein the two compositions are mixed before applying on the skin of a patient in need thereof, thus administering a combination product.

In one aspect, this invention provides a regimen of administration comprising a once daily or twice daily administration of a therapeutically effective dose of a first composition comprising from about 2% w/w to about 10% w/w benzoyl peroxide, and a second composition comprising from about 0.5% w/w to about 10% w/w of minocycline or a pharmaceutically acceptable salt thereof, wherein the two compositions are concomitantly administered from a dual chamber dispenser or from two separate dispensers and mixed before applying on the skin of a patient in need thereof for up to 2 weeks, up to 1 month, preferably up to 2 months and more preferably up to 3 months.

In one aspect, this invention provides a method of treating, preventing, or inhibiting acne or rosacea comprising administering to a subject in need thereof between 2% w/w to about 10% w/w benzoyl peroxide and from about 0.25% w/w to about 5% minocycline or a pharmaceutically acceptable salt thereof, wherein the effect of the combination is superior than each of its individual components when administered alone.

In one aspect, this invention provides a method of treating, preventing, or inhibiting acne or rosacea comprising administering to a subject in need thereof between 2% w/w to about 10% w/w benzoyl peroxide and from about 0.25% w/w to about 5% minocycline or a pharmaceutically acceptable salt thereof, wherein the benzoyl peroxide and the minocycline are co-administered by administration of a single combination composition, or by separate administration of (i) a first composition comprising said benzoyl peroxide followed by administration of a second composition comprising said minocycline or a pharmaceutically acceptable salt thereof; or (ii) a second composition comprising said minocycline or a pharmaceutically acceptable salt thereof followed by administration of a first composition comprising said benzoyl peroxide.

In one aspect, this invention provides a kit, having a first chamber charged with a first composition comprising from about 2% w/w to about 10% w/w benzoyl peroxide, and a second chamber charged with a second composition comprising from about 0.5% w/w to about 10% w/w of minocycline or a pharmaceutically acceptable salt thereof, wherein the two compositions are mixed before applying on the skin of a patient in need thereof, thus administering a combination product; or administering the first and second composition sequentially.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 shows a degradation of minocycline in the presence of non-encapsulated (straight line) and encapsulated benzoyl peroxide (dashed line).

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

In one embodiment, the present invention provides methods, a dual chamber dispenser, a kit and regimens of administration for acne and rosacea treatment, comprising concomitant topical administration of benzoyl peroxide (BPO) and minocycline.

In one embodiment, the present invention provides methods of treatment of an acne patient in need thereof by the concomitant topical administration of BPO and minocycline.

In one embodiment, the present invention provides methods of treatment of a rosacea patient in need thereof by the concomitant topical administration of BPO and minocycline.

The concomitant once daily or twice daily topical administration may be done by administration of a single composition comprising both benzoyl peroxide and minocycline, or alternatively, by topical administration of a first composition comprising BPO and a second composition comprising minocycline, concomitantly (same time) administered once daily or twice daily from a dual chamber dispenser or from two separate dispensers and mixed before applying on the skin of a patient in need thereof.

In one embodiment, the methods, dual chamber dispenser, kit and regimens of administration for acne and rosacea treatment comprise a first composition comprising between about 2% w/w to about 12% w/w benzoyl peroxide; and a second composition comprising between about 0.5% w/w to about 10% minocycline. In another embodiment, the benzoyl peroxide is encapsulated.

In one embodiment, the methods, dual chamber dispenser, kit and regimens of administration for acne and rosacea treatment comprise a first composition comprising between about 2% w/w to about 10% w/w benzoyl peroxide; and a second composition comprising between about 0.5% w/w to about 10% minocycline. In another embodiment, the benzoyl peroxide is encapsulated.

In one embodiment this invention provides a method of treating, preventing, or inhibiting acne or rosacea comprising administering to a subject in need thereof between 2% w/w to about 10% w/w benzoyl peroxide and from about 0.25% w/w to about 5% minocycline or a pharmaceutically acceptable salt thereof, wherein the benzoyl peroxide and the minocycline are co-administered by administration of a single combination composition, or by separate administration of (i) a first composition comprising benzoyl peroxide followed by administration of a second composition comprising minocycline or a pharmaceutically acceptable salt thereof; or (ii) a second composition comprising minocycline or a pharmaceutically acceptable salt thereof followed by administration of a first composition comprising benzoyl peroxide; or (iii) the first composition and the second composition are concomitantly administered. In another embodiment, the benzoyl peroxide is encapsulated. In another embodiment, the two compositions are mixed before applying on the skin of a patient in need thereof, thus administering a combination product. In another embodiment, the first composition comprises 10% BPO and the second composition comprises 3% w/w minocycline or a pharmaceutically acceptable salt thereof.

In one embodiment this invention provides a method of treating, preventing, or inhibiting acne or rosacea comprising administering to a subject in need thereof between 2% w/w to about 12% w/w benzoyl peroxide and from about 0.25% w/w to about 5% minocycline or a pharmaceutically acceptable salt thereof, wherein the benzoyl peroxide and the minocycline are co-administered by administration of a single combination composition, or by separate administration of (i) a first composition comprising benzoyl peroxide followed by administration of a second composition comprising minocycline or a pharmaceutically acceptable salt thereof; or (ii) a second composition comprising minocycline or a pharmaceutically acceptable salt thereof followed by administration of a first composition comprising benzoyl peroxide; or (iii) the first composition and the second composition are concomitantly administered. In another embodiment, the benzoyl peroxide is encapsulated. In another embodiment, the two compositions are mixed before applying on the skin of a patient in need thereof, thus administering a combination product. In another embodiment, the first composition comprises 10% BPO and the second composition comprises 3% w/w minocycline or a pharmaceutically acceptable salt thereof.

In one embodiment, the methods and the regimen of administration of this invention comprise mixing a first composition and a second composition before applying on the skin. It is understood, that when applying the combination of the first and second compositions, the concentration of the active agents (i.e BPO and minocycline) is reduced according the volume of each composition used in the mixture. According to another embodiment, if not indicated otherwise, the combinations used include equal volumes of the first and second compositions. Thus, for example, if the first composition comprises 10% BPO and the second composition comprises 3% minocycline, upon mixing equal volume of the two compositions, 5% BPO and 1.5% minocycline are applied on the skin.

As used herein the term “a regimen for the treatment of acne or rosacea” is used herein interchangeably with the term “method of treating acne or rosacea” having all the same meaning and qualities. The term “regimen” as used herein should be understood to relate to a medical treatment regimen regulating the treatment of acne or rosacea in a subject suffering therefrom, including the regulation of the medicament administered (fixed dose combination of the active agents: minocycline or a pharmaceutically acceptable salt thereof and BPO), the frequency of administration (i.e. once a day), the duration of treatment (i.e. up to 12 weeks), the method of administration (i.e. topical) and the location of administration (i.e. topically applying onto an affected skin area).

When relating to the treatment of “acne” it should be understood to relate to the treatment of a skin condition or disease also known as acne vulgaris in any form or place of its occurrence or severity (mild, moderate, severe or any combinations thereof. In some subjects parts of area of the skin may be mildly inflicted while other area of the skin of the same individual may be severely inflicted). Mild acne is classically defined as open (blackheads) and closed (whiteheads) clogged skin follicles (comedones) limited to the face with occasional inflammatory lesions. Acne may be considered to be of moderate severity when a higher number of inflammatory papules and pustules occur on the skin. Severe acne is said to occur when nodules are the characteristic facial lesions, and involvement of other areas of the body is extensive. Inflammatory acne lesions include papule lesions (small, solid elevation less than 5 mm in diameter, most of the lesion is above the surface of the skin), pustule lesions (small circumscribed elevation less than 5 mm in diameter that contains yellow-white exudate), nodule lesions (inflammatory lesion greater than or equal to 5 mm in diameter) and cyst lesions (inflammatory lesion that contains yellow-white exudate that is greater than or equal to 5 mm in diameter). Non-inflammatory lesions include open comedone (black head) (lesion in which the follicle opening is widely dilated with the contents protruding out onto the surface of the skin, with compacted melanin cells giving the plug a black appearance) and closed comedone (white head) (lesion in which the follicle opening is closed, but the sebaceous gland is enlarged by the pressure of the sebum buildup, which in turn cause the skin around the follicle to thin and become elevated with a white appearance).

When relating to the treatment of “rosacea” it should be understood to relate to the treatment of the four stages of rosacea which develops over several years, in spasms aggravated by variations in temperature, alcohol, spices, exposure to sunlight and stress.

The various stages of the disease are the following:

Stage 1: stage of erythema episodes. The patients have erythrosis spasms due to the sudden dilation of the arterioles of the face, which then take on a congestive, red appearance. These spasms are caused by the emotions, meals and temperature changes.

Stage 2: stage of couperosis, i.e., of permanent erythema with telangiectasia. Certain patients also have oedema on the cheeks and the forehead.

Stage 3: inflammatory stage (papularpostular rosacea) with appearance of inflammatory papules and pustules, but without affecting the sebaceous follicles and thus with absence of cysts and comedones.

Stage 4: rhinophyma stage. This late phase essentially affects men. The patients have a bumpy, voluminous red nose with sebaceous hyperplasia and fibrous reordering of the connective tissue.

In some embodiments, said rosacea is papularpostular rosacea (i.e. inflammatory rosacea, see Rapini, Ronald P. et al. (2007). Dermatology: 2-Volume Set. St. Louis: Mosby and James, William et al. (2005). Andrews' Diseases of the Skin: Clinical Dennatology. (10th ed.). Saunders p. 245).

Dispensing the BPO and Minocycline Compositions from a Dual-Chamber Dispenser

In one embodiment, the compositions consisting of a first composition comprising BPO and a second composition comprising minocycline, concomitantly administered once daily or twice daily from a dual chamber dispenser.

The dual chamber dispenser may be disposable (for one use) or for multiple uses.

The dual chamber dispenser may be a commercially available device or a dispenser custom manufactured for the compositions of this invention, made of materials resistant to the two actives and the compositions of this invention and having chamber volumes fit for the methods of treatment and regimens of administration disclosed herein.

Such dual chamber dispensers have been disclosed, for example, in U.S. Pat. No. 6,117,433 or U.S. Patent Application No. 2004/0157766 (now abandoned).

In one embodiment, this invention provides a dual chamber dispenser, having a first chamber charged with a first composition comprising from about 2% w/w to about 10% w/w benzoyl peroxide, and a second chamber charged with a second composition comprising from about 0.5% w/w to about 10% w/w of minocycline or a pharmaceutically acceptable salt thereof, wherein the two compositions are mixed before applying on the skin of a patient in need thereof, thus administering a combination product.

Dispensing the BPO and minocycline compositions, instantly mixed on the skin of a patient from two separate dispensers, one containing a BPO composition and the other a minocycline composition.

This concomitant administration of BPO and minocycline is carried out by application to the skin of a patient an instantly mixed composition from two separate dispensers, one containing a BPO composition and the other a minocycline composition. The two compositions are instantly mixed on the skin of the patient.

The methods for acne and rosacea treatment of this invention comprise concomitant or sequential topical administration of benzoyl peroxide (BPO) and minocycline for up to 2 weeks, up to 1 month, preferably up to 2 months and more preferably up to 3 months.

In one embodiment, this invention provides a method of treating, preventing, or inhibiting acne or rosacea comprising administering to a subject in need thereof between 2% w/w to about 10% w/w benzoyl peroxide and from about 0.25% w/w to about 5% minocycline or a pharmaceutically acceptable salt thereof through a dual chamber dispenser of this invention, wherein the effect of the combination is superior than each of its individual components when administered alone.

In one embodiment, this invention provides a method of treating, preventing, or inhibiting acne or rosacea comprising administering to a subject in need thereof between 2% w/w to about 12% w/w benzoyl peroxide and from about 0.25% w/w to about 5% minocycline or a pharmaceutically acceptable salt thereof through a dual chamber dispenser of this invention, wherein the effect of the combination is superior than each of its individual components when administered alone.

The regimens of administration for acne and rosacea treatment of this invention comprise once daily or twice daily concomitant or sequential application of the compositions of this invention on the skin of a patient in need thereof for a period of up to 2 weeks, up to 1 month, preferably up to 2 months and more preferably up to 3 months.

In one embodiment, this invention provides a regimen of administration comprising a once daily or twice daily administration a therapeutically effective dose of a first composition comprising from about 2% w/w to about 10% w/w benzoyl peroxide, and a second composition comprising from about 0.5% w/w to about 10% w/w of minocycline or a pharmaceutically acceptable salt thereof, wherein the two compositions are concomitantly administered from a dual chamber dispenser or from two separate dispensers and mixed before applying on the skin of a patient in need thereof for up to 2 weeks, up to 1 month, preferably up to 2 months and more preferably up to 3 months.

In one embodiment, this invention provides a regimen of administration comprising a once daily or twice daily administration a therapeutically effective dose of a first composition comprising from about 2% w/w to about 12% w/w benzoyl peroxide, and a second composition comprising from about 0.5% w/w to about 10% w/w of minocycline or a pharmaceutically acceptable salt thereof, wherein the two compositions are concomitantly administered from a dual chamber dispenser or from two separate dispensers and mixed before applying on the skin of a patient in need thereof for up to 2 weeks, up to 1 month, preferably up to 2 months and more preferably up to 3 months.

In another embodiment, the regimen of administration comprises mixing the two compositions before applying on the skin of a patient in need thereof, thus administering a combination product. In another embodiment, the first composition comprises 10% BPO and the second composition comprises 3% w/w minocycline or a pharmaceutically acceptable salt thereof.

A Kit Comprising BPO and Minocycline Compositions

In one embodiment, this invention provides a kit comprising a first composition comprising from about 2% w/w to about 10% w/w benzoyl peroxide, and a second composition comprising from about 0.5% w/w to about 10% w/w of minocycline or a pharmaceutically acceptable salt thereof, wherein the two compositions are mixed before applying on the skin of a patient in need thereof, thus administering a combination product; or administering the first and second composition sequentially.

In yet another aspect, the kit provides a packaged product, comprising a sealable container and a composition as described herein that is contained within the sealable container. The sealable container can have many different configurations, e.g., including but not limited to the various types of containers that are used for packaging cream, gel and ointment products for consumer use. Non-limiting examples of suitable sealable containers include pump-type bottles, nozzle-type bottles, tubes, sachets, packets, and various other configurations known to those skilled in the art.

Compositions Used in the Methods, Dual Chamber Dispenser, Kit and Regimen Administration of this Invention

In one embodiment, the methods of this invention make use of a first composition comprising between about 2% w/w to about 10% w/w benzoyl peroxide in combination with a second composition comprising from about 0.5% w/w to about 10% w/w of minocycline.

In one embodiment, the methods of this invention make use of a first composition comprising between about 2% w/w to about 12% w/w benzoyl peroxide in combination with a second composition comprising from about 0.5% w/w to about 10% w/w of minocycline.

In one embodiment, the dual chamber dispenser, the kit and the regimen administration of this invention comprise a first composition comprising between about 2% w/w to about 10% w/w benzoyl peroxide in combination with a second composition comprising from about 0.5% w/w to about 10% w/w of minocycline.

In another embodiment, the first composition comprises 2%, 3%, 4%, 5%, 6%, 7%, 8%, 9% or 10% by weight benzoyl peroxide. In another embodiment, the first composition comprises between 2% to 6% by weight benzoyl peroxide. In another embodiment, the first composition comprises between 5% to 10% by weight benzoyl peroxide. In another embodiment, the benzoyl peroxide is encapsulated, coated, adsorbed, embedded, impregnated, dispersed, entrapped, or encased in a polymeric material and providing a sustained release formulation. In another embodiment, the benzoyl peroxide is encapsulated.

In another embodiment, the second composition comprises 0.5, 1%, 2%, 3%, 4%, 5%, 6%, 7%, 8%, 9% or 10% by weight minocycline or its pharmaceutically acceptable salt thereof. In another embodiment, the second composition comprises between 0.5% to 5% by weight minocycline or its pharmaceutically acceptable salt thereof. In another embodiment, the second composition comprises between 2% to 10% by weight minocycline or its pharmaceutically acceptable salt thereof. In another embodiment, the minocycline salt is a minocycline hydrochloride.

In one embodiment, the second composition (minocycline) is anhydrous.

In one embodiment, the first (BPO)) and second (minocycline) compositions are the same dosage forms or different. In another embodiment, the dosage forms are selected from a cream, a lotion, a gel, an ointment, an emulsion, a solution, a suspension, an elixir, a tincture, a paste, a foam, an aerosol or a spray. In another embodiment, the first and second compositions are both cream, lotion, ointment or a gel. In another embodiment, the first composition dosage form is a cream and second composition dosage form is an ointment.

The compositions of this invention are pharmaceutical compositions comprising carriers or vehicles suitable for administration of the compounds provided herein including any carriers known to those skilled in the art to be suitable for the particular mode of administration. In addition, the compounds may be formulated as the sole pharmaceutically active ingredient in the composition or may be combined with other active ingredients. The active agents are included in the carrier in an amount sufficient to exert a therapeutically useful effect i.e., amelioration of the symptoms of rosacea or acne, with minimal or no toxicity or other side effects. Generally, emollient or lubricating vehicles that help hydrate the skin are more preferred than volatile vehicles, such as ethanol, that dry the skin. Examples of suitable bases or vehicles for preparing compositions for use with human skin are petrolatum, petrolatum plus volatile silicones, lanolin, cold cream and hydrophilic ointment.

Suitable pharmaceutically and dermatologically acceptable vehicles for topical application include those suited for use include lotions, creams, foams, solutions, gels, tapes and the like. Generally, the vehicle is either organic in nature or an aqueous emulsion and capable of having the selected compound or compounds, which may be micronized, dispersed, suspended or dissolved therein. The vehicle may include pharmaceutically-acceptable emollients, moisturizers, including lactic acid, ammonium lactate and urea, skin penetration enhancers, coloring agents, fragrances, emulsifiers, thickening agents, vegetable oils, essential oils, zinc oxide and solvents.

Benzoyl Peroxide Composition (Referred in this Invention as the First Composition)

According to some embodiments of the present invention, the coated form of the benzoyl peroxide (microcapsule) may be in form of a polymeric microsponge/silica microsphere where the benzoyl peroxide is adsorbed, embedded, impregnated or entrapped in the microsponge/silica microsphere as described for example in U.S. Pat. Nos. 4,690,825; 5,145,675, 5,879,716, 5,955,109, and 9,452,137 incorporated herein by reference in their entirety.

In other embodiments, microcapsules are formed by the encapsulation process disclosed in the following publications (herein incorporated by reference): U.S. Pat. Nos. 7,629,394, 9,205,395, US 2015/0328615, US 2014/0186630. Controlled release microcapsules: IN01958CH2007, IN02080CH2007, U.S. Pat. Nos. 4,235,872, 4,670,250, EP 0248531, U.S. Pat. Nos. 4,970,031, 5,238,714, WO9321764, U.S. Pat. No. 5,575,987, WO9420075, US 2004/137031, US 2006/003014, US 2010/180464.

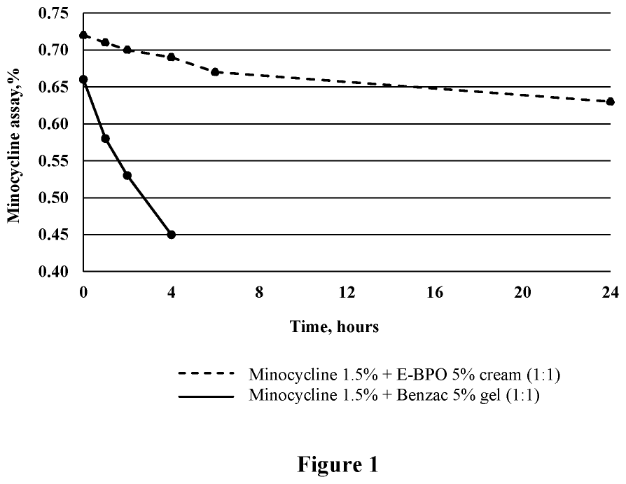

Mixing a first composition comprising non-encapsulated BPO with a second composition comprising minocycline as described in Example 5, Example 6 and demonstrated in FIG. 1, demonstrate degradation of the minocycline after 4 hrs. Surprisingly, it was found that mixing encapsulated BPO with minocycline demonstrated stability of the minocycline for at least 24 hrs.

Some embodiments provide a first composition for topical application, where the composition contains: microcapsules having a core that comprises BPO and a shell that comprises an inorganic polymer. The composition can be in a variety of forms, including, but not limited to, an emulsion form, a cream form, an aqueous solution form, an oil form, an ointment form, a paste form, a gel form, a lotion form, and a suspension form. In some embodiments, the microcapsules having a core that comprises BPO can be in the form of an emulsion prior to formation of the composition. In further embodiments, these emulsions may be incorporated into a cream, gel, lotion, or other form providing the composition described above.

### Microcapsules

As used herein, the term “microcapsule” refers to any micro- or nano-sized particle having a core-shell structure that is capable of encasing, encapsulating or entrapping compounds, including but not limited to active ingredients such as BPO. In some embodiments, microcapsules are made by a sol-gel process, e.g., as generally described in WO 03/034979 and WO 2011/080741.

### Core

As used herein, the term “core” refers to the inside part of a microcapsule comprising at least one active ingredient surrounded by a shell of the microcapsule. In some embodiments, the core can be solid at room temperature. In other embodiments, the core can be in a semi-solid phase at room temperature. In some embodiments, the core can be in the form of an emulsion, for example an oil-in-water emulsion. In some embodiments, the core can be in the form of oil solution. In some embodiments, the core can be in the form of an aqueous solution. In some embodiments, the core can be in the form of a dispersion.

Additional compound(s) can be present in the core. Non-limiting examples of the additional compounds that can be present in the core include phase changing materials (PCMs), carriers, excipients, antioxidants, pharmaceutically acceptable polymers, and salts. In some embodiments, the core comprises at least one phase changing material. Exemplary phase changing materials include, but are not limited to, natural and synthetic paraffins; C₁₀-C₁₀₀ (straight, branched, and cyclic) alkanes, alkenes and alkynes; C₁₀-C₁₀₀ aliphatic alcohols (e.g., fatty alcohols); fatty acids; carnauba wax; beeswax; and mixtures thereof. In some embodiments, the core comprises at least one antioxidant. Examples of antioxidants include, but are not limited to, butylated hydroxytoluene (BHT), butylated hydroxyanisole (BHA), vitamin E, vitamin E acetate, vitamin E palmitate, vitamin C, an ester of vitamin C, and one or more salts of vitamin C.

### Shell

As used herein, the term “shell” refers to the part of a microcapsule that surrounds the core of the microcapsule. In some embodiments, the shell comprises an inorganic polymer (for example, a silica polymer). In some embodiments, the inorganic polymer can be prepared from a sol-gel precursor.

As used herein, the term “sol-gel precursor” refers to any metal or semi-metal organo-metallic monomer, or a prepolymer (which means several monomers polymerized together) thereof, which provide a glass or ceramic material by in-situ polymerization (an inorganic sol-gel polymerization process). In some embodiments, the sol-gel precursor can be a metal or semi-metal organo-metallic monomer. Examples of sol-gel precursor include, but are not limited to, a metal alkoxide monomer; a semi-metal alkoxide monomer; a metal ester monomer; a semi-metal ester monomer; a silazane monomer; a colloidal silica; a monomer of the formula M(R)ₙ(P)ₘ, where M can be a metallic or a semi-metallic element, R can be a hydrolyzable substituent, n can be an integer from 2 to 6, P can be a non polymerizable substituent, and m can be an integer from 0 to 6; and a partially hydrolyzed and partially condensed polymer thereof. Various metallic or semi metallic elements can be used in the sol-gel precursor, for example, Si, Ti, Zr, Al, and Zn. Examples of semi-metal alkoxide monomers include, but are not limited to, tetramethoxysilane (also known as tetramethyl orthosilicate or TMOS), tetraethoxysilane (also known as tetraethyl orthosilicate or TEOS), dimethyldimethoxysilane, methyltrimethoxysilane, diethyldimethoxysilane, and sodium silicate.

In some embodiments, the sol-gel precursor can be selected from a silicon alkoxide monomer; a silicon ester monomer; a monomer of the formula Si(R)ₙ(P)ₘ, wherein R can be a hydrolyzable substituent, n can be an integer from 2 to 4, P can be a non polymerizable substituent, and m can be an integer from 0 to 4; a partially hydrolyzed and partially condensed polymer of any of the above, and mixtures of any of the above. Non-limiting examples of silicon alkoxide monomer include tetramethoxy silane, tetraethoxy silane, and combinations thereof. Non-limiting examples of monomers of the formula Si(R)n(P)m include methyl trimethoxysilane, dimethyl dimethoxysilane, and combinations thereof.

In one embodiment, the first composition comprises a first core-shell microcapsules comprising a first core that comprises benzoyl peroxide and a first shell that comprises a first silica polymer, the benzoyl peroxide being present in the composition in an initial amount of about 10% by weight, based on the total weight of the first composition.

Minocycline Composition (Referred in this Invention as the Second Composition)

In one aspect the methods, regimen of administration, dispenser or kit make use of a second composition comprising minocycline. In another embodiment, the second composition comprises:
from about 0.5% w/w to about 10% w/w of minocycline or pharmaceutical acceptable salt thereof;
from about 60% to about 90% by weight of the composition of at least one hydrophobic oil, and
from about 5% to about 25% by weight of the composition of at least one fatty alcohol;
wherein the at least one hydrophobic oil and the at least one fatty alcohol comprise in total from about 65% to about 99% by weight of the composition;
wherein the at least one hydrophobic oil and the at least one fatty alcohol are in a weight ratio of from about 4:1 to about 8:1; and
wherein the composition is essentially free of water, waxes, fatty acids, shea butter, short chain alcohols, polyols, polar solvents, polymers, hydrocarbon-based oils, mineral oils and petrolatum.

In some other embodiments, the at least one hydrophobic oil in the compositions of the second composition is selected from the group consisting of a therapeutic oil, an alexandria laurel tree oil, an almond oil, an essential oil, an unsaturated or polyunsaturated oil, an apricot stone oil, an avocado oil, a barley oil, a basil oil, a borage seed oil, a calendula oil, a camphor oil, a candle nut tree oil, a canola oil, a cardamom oil, a carrot oil, a castor oil, a citronella oil, a clary sage oil, a clove oil, a coconut oil, a cod-liver oil, a corn oil, a cotton oil, a cottonseed oil, a cypress oil, a cyclomethicone oil, an epoxy-modified silicone oil, an ester oil, an evening primrose oil, a fatty acid-modified silicone oil, a flaxseed oil, a fluoro group-modified silicone oil, a frankincense oil, a ginger oil, a grape seed oil, a grapefruit oil, a groundnut oil, a hazelnut oil, a hempseed oil, a herring oil, a hyssop oil, a jasmine oil, a jojoba oil, a lavender oil, a lemon oil, a lucerne oil, a maize germ oil, a maleated soybean oil, a mandarin oil, a manuka oil, a marjoram oil, a marrow oil, a MCT oil, a millet oil, a myrrh oil, a neroli oil, a nutmeg oil, oils from animal origin, oils of plant origin, an olive oil, a palm oil, a passionflower oil, a peanut oil, a petitgrain oil, a polyether group-modified silicone oil, a poppy oil, a rapeseed oil, a rosehip oil, a rye oil, a safflower oil, a sage oil, a salmon oil, a sesame oil, a silicone oil, a soybean oil, a soybean oil, a sunflower oil, a sweet almond oil, a sysymbrium oil, a syzigium aromaticum oil, a tangerine oil, a tea tree oil, unsaturated or polyunsaturated oils, a vanilla oil, a verbena oil, a walnut oil, a wheat germ oil, and mixtures of any two or more thereof.

In some other embodiments, the at least one fatty alcohol in the composition of this invention is selected from myristyl alcohol, cetyl alcohol, behenyl alcohol and mixtures thereof.

In addition to the above ingredients, the minocycline composition of this invention may further comprise from about 0.1% to about 20% by weight of the composition of at least one fatty acid selected from stearic acid, palmitic acid and mixtures thereof, in which case it may also contain a polymer. Exemplary polymers are selected from the group consisting of a polypropylene glycol, polyethylene glycol, ethylcellulose, alkylated guar gum, trimethylsiloxysilicate, alkyl-modified silicone, polyamide-modified silicone homopolymers and copolymers of alkyl methacrylates, alkyl acrylates and alkyl styrenes, polyisobutene, polybutyl methacrylate and polycyclohexylstyrene.

As used herein, the term “topical” application refers to an application onto the skin, hair, ears, and/or mucous membranes.

Some embodiments disclosed herein provide a first composition for topical application, wherein the composition comprises: a plurality of first microcapsules having a core that comprises benzoyl peroxide and a shell that comprises an inorganic polymer.

In some embodiments, the non-ionic polymer can be present in an amount effective to provide viscosity stabilization. In some embodiments, the viscosity stabilization can be effective to maintain the viscosity of the composition at more than about 20,000 cps, about 25,000 cps, about 30,000 cps, about 35,000 cps, about 40,000 cps, about 45,000 cps, or about 50,000 cps as measured after manufacture followed by 3 months storage at a storage temperature. The storage temperature can be about 5° C. or about 25° C. In some embodiments, the non-ionic polymer can be present in an amount effective to provide degradation stabilization.

In certain embodiments, the non-ionic polymer can be present in an amount effective to provide the viscosity stabilization and degradation stabilization. Other non-ionic polymers and effective amounts thereof that provide the viscosity stabilization and/or the degradation stabilization may be identified by those skilled in the art using routine experimentation guided by the teachings provided herein. Non-limiting examples of suitable non-ionic polymers include polyvinyl pyrrolidone (PVP), polyvinyl pyrrolidone-co-vinyl acetate, polyamide, polyurethane, polyurea, and mixtures thereof.

In another aspect, the present disclosure provides methods of preparing a composition comprising microcapsules disclosed herein. Those skilled in the art will appreciate the manner in which the working examples set forth below provide a specific description of how to make particular compositions and components thereof. Those skilled in the art will also appreciate the manner in which the specific working examples can be generalized and adapted to produce the other compositions described herein and components thereof.

In yet another aspect, the present disclosure provides a method for treating acne and rosacea. In this context, terms such as “treat,” “treating,” “treatment,” etc. include inhibiting the surface condition (e.g., by arresting its development), relieving the surface condition (e.g., causing regression) and/or relieving one or more conditions caused by the surface condition (e.g., reducing one or more symptoms). Effective amounts of the compositions described herein for treating various surface conditions can be determined by those skilled in the art in the usual manner, e.g., by clinical trials, with appropriate adjustments by skilled clinicians in individual cases.

In some embodiments, the minocycline salt is a minocycline HCl salt.

The following examples are presented in order to more fully illustrate the disclosed embodiments. They should in no way be construed, however, as limiting the broad scope of the disclosure.

### EXPERIMENTAL SECTION

### Example 1. Preparation of Encapsulated Benzoyl Peroxide (BPO) (15% E-BPO Water Suspension)

A) Preparation of Benzoyl Peroxide Dispersion and Acid Cocktail

A benzoyl peroxide dispersion was prepared by mixing 378 grams of CTAC CT-429 (Cetrimonium Chloride 30%), 9020 grams of hydrous benzoyl peroxide (75%), and 16600 grams water under high shear. The dispersion was homogenized for 60 min at 33° C. (no more than 45° C.). An acid cocktail was prepared using 1013 grams Hydrochloric Acid (37%), 215.3 grams anhydrous Citric Acid, 322.3 grams Lactic Acid (90%), and 1632 grams water.

B) Coating Cycle

The coating cycle was started by adding 953 grams sodium silicate solution extra pure (28%) to the benzoyl peroxide dispersion prepared in step a) under high shear, followed by adding the acid cocktail prepared in step (a) and followed by adding 1675 grams PDAC (3%) solution to the mixture. The cycle was repeated for additional 5 times. After the 6^{th} cycle, the pH of the mixture was adjusted to 5.0 using the acid cocktail, and water was added to complete the total weight of the mixture to 45 kilograms.

The composition of the final BPO water suspension product is shown in Table 1.

TABLE 1
Composition of the encapsulated BPO 15% water suspension

| Ingredient | % w/w of ingredient in the suspension |
|---|---|
| Polyquarterniurn-7 | 5.6 |
| Hydrochloric Acid 37% | 2.0 |
| Citric Acid, Anhydrous | 0.4 |
| Lactic Acid | 0.6 |
| Silicone Dioxide | 3.4 |
| Sodium Hydroxide | 1.4 |
| Cetrimonium Chloride | 0.84 |
| Hydrous Benzoyl Peroxide | 15.00 |
| Sterile Water for Irrigation | Up to 100% |

### Example 2. Preparation of Formulation of Encapsulated BPO (5%) Cream

Oil Phase: 32 grams of Cyclomethicone 5-NF, 24 grams of Cetyl Alcohol, 16 grams of Polyoxyl 100 Stearate, and 24 grams of Glyceryl Monosterate were mixed at 65-70° C.

Water phase: A solution of 0.8 grams of Ethylenediaminetetraacetate disodium salt, 32 grams of glycerin (99.5%), 4 grams of phenoxyethanol and 320 grams of water was mixed and heated to 65-70° C.

The oil phase was added to the water phase under high shear at 65-70° C., and the resulting emulsion was homogenized 17000 rpm for 10 minutes. 3.2 grams of Citric Acid and 275.9 grams of encapsulated BPO 15% water suspension made as described in Example 1 were mixed and heated to 45° C. The resulting mixture was added to the emulsion at 60-70° C. and mixed at 150 rpm for 10 minutes. The pH of the emulsion was adjusted to 4 using HCl 10%.

The emulsion was stirred at 150 rpm for 10 minutes and then water was added until the total weight of the emulsion reached 800 grams. The composition of the formulation prepared in this example is shown in Table 2.

TABLE 2
Composition of E- BPO 5% formulation

| | % w/w of ingredient in the |
|---|---|
| Ingredient | composition |
| E-BPO, 15% suspension | 33.33 |
| Polyoxyl 100 stearate | 2.0 |
| Cetyl alcohol | 3.0 |
| Cyclomethicone 5- NF | 4.0 |
| Glyceryl Mono and Di stearate | 3.0 |
| Hydrochloric Acid 10% | pH adjustment |
| Citric Acid, Anhydrous | 0.4 |
| Sodium Hydroxide Pellets | pH adjustment |
| Ethylenediaminetetraacetate Disodium salt | 0.1 |
| Glycerin 99.5% | 4.0 |
| Phenoxyethanol | 0.5 |
| Purified water or higher grade | Up to 100 |

### Example 3. Preparation of Formulation of Minocycline 1.5% Ointment

In a 1 L beaker, 7.2 grams of light mineral oil, 25 grams of cyclomethicone, 124.55 grams of coconut oil, 256.5 grams of soybean oil, 10 grams of hydrogenated castor oil, 10 grams of beeswax, 12.5 grams of myristyl alcohol, 17.5 grams of cetostearyl alcohol, 7.5 grams of stearyl alcohol, 5.5 grams of behenyl alcohol, and 15 grams of stearic acid were added, mixed and heated using a water bath to 70° C. until all the ingredients were dissolved. Then 1.25 grams of Aerosil (SiO₂) was added and the mixture was mixed for 10 minutes. The mixture was cooled to 50-60° C., and then 7.5 grams of minocycline HCl was added, followed by high shear mixing at 7000 rpm for 5 min. The reaction mixture was cooled fast to room temperature.

TABLE 3
Composition of minocycline 1.5% formulation

| Ingredients | Concentration (%) |
|---|---|
| Light mineral oil | 1.44 |
| Cyclomethicone | 5.00 |
| Coconut oil | 24.91 |
| Soybean oil | 51.30 |
| Hydrogenated castor oil | 2.00 |
| Beeswax (solid wax) | 2.00 |
| Myristyl alcohol | 2.50 |
| Cetostearyl alcohol | 3.50 |
| Stearyl alcohol | 1.50 |
| Behenyl alcohol | 1.10 |
| Aerosil (SiO₂) | 0.25 |
| Stearic acid | 3.00 |
| Minocycline HCl | 1.5 |

### Example 5. Preparation of a Combination of Minocycline and Non-Encapsulated Benzoyl Peroxide

In a 150 mL beaker, 25 grams of commercially available non-encapsulated benzoyl peroxide-Benzac 5% (Galderma, France)) and 25 grams of minocycline 1.5% ointment (prepared as described in Example 3 above) were manually mixed with a spatula for 3 minutes to obtain a homogeneous mixture. Three portions of 2 g of the mixture were taken for analysis of the minocycline assay at time zero, 1 h, 2 h and 4 h, at 25° C. The testing after 6 h and 24 h were not conducted because of the high degradation of minocycline after 4 h at 25° C., as can be seen in FIG. 1. Before each test/sampling, the mixture was manually mixed for 1 minute.

FIG. 1 shows a degradation of minocycline in the presence of non-encapsulated and encapsulated benzoyl peroxide.

### Example 6. Preparation of a Combination of Minocycline and Encapsulated Benzoyl Peroxide

In a 150 mL beaker, 25 grams of encapsulated BPO 5% cream (prepared as described in Example 2 above) and 25 grams of minocycline 1.5% ointment (prepared as described in Example 3 above) were manually mixed with a spatula for 3 minutes to obtain a homogeneous mixture. Three portions of 2 g of the mixture were taken for analysis of the minocycline assay at time zero, 1 h, 2 h, 4 h, 6 h and 24 h, at 25° C. Before each test/sampling, the mixture was manually mixed for 1 minute.

FIG. 1 demonstrates the stability of the combination of minocycline and encapsulated benzoyl peroxide for at least 24 hrs.

While certain features have been illustrated and described herein, many modifications, substitutions, changes, and equivalents will now occur to those of ordinary skill in the art. It is, therefore, to be understood that the appended claims are intended to cover all such modifications and changes as fall within the true spirit of this disclosure.

1. A dual chamber dispenser, having a first chamber charged with a first composition comprising from about 2% w/w to about 10% w/w benzoyl peroxide, and a second chamber charged with a second composition comprising from about 0.5% w/w to about 10% w/w of minocycline or a pharmaceutically acceptable salt thereof, wherein the two compositions are mixed before applying on the skin of a patient in need thereof, thus administering a combination product.

2. The dual chamber dispenser of claim 1, wherein the first composition comprises 10% w/w benzoyl peroxide, and the second composition comprises 3% w/w minocycline or a pharmaceutically acceptable salt thereof, wherein the two compositions are mixed before applying on the skin of a patient in need thereof, thus administering a combination product.

3. The dual chamber dispenser of claim 1, wherein the first and second compositions are the same dosage forms or different.

4. The dual chamber dispenser of claim 3, wherein the dosage forms are selected from a cream, a lotion, a gel, an ointment, an emulsion, a solution, a suspension, an elixir, a tincture, a paste, a foam, an aerosol or a spray.

5. The dual chamber dispenser of claim 3, wherein the first and second compositions are both cream, lotion, ointment or a gel.

6. The dual chamber dispenser of claim 3, wherein the first composition dosage form is a cream and second composition dosage form is an ointment.

7. The dual chamber dispenser of claim 1, wherein the benzoyl peroxide is optionally encapsulated.

8. The dual chamber dispenser of claim 1, wherein the benzoyl peroxide is encapsulated, coated, adsorbed, embedded, impregnated, dispersed, entrapped, or encased in a polymeric material and providing a sustained release formulation.

9. The dual chamber dispenser of claim 1, wherein the minocycline is minocycline HCl.

10. The dual chamber dispenser of claim 1, wherein the second composition is anhydrous.

11. A regimen of administration comprising a once daily or twice daily administration of a therapeutically effective dose of a first composition comprising from about 2% w/w to about 10% w/w benzoyl peroxide, and a second composition comprising from about 0.5% w/w to about 10% w/w of minocycline or a pharmaceutically acceptable salt thereof, wherein the two compositions are concomitantly administered from a dual chamber dispenser or from two separate dispensers and mixed before applying on the skin of a patient in need thereof for up to 2 weeks, up to 1 month, preferably up to 2 months and more preferably up to 3 months.

12. A method of treating, preventing, or inhibiting acne or rosacea comprising administering to a subject in need thereof between 2% w/w to about 10% w/w benzoyl peroxide and from about 0.25% w/w to about 5% minocycline or a pharmaceutically acceptable salt thereof, wherein the effect of the combination is superior than each of its individual components when administered alone.

13. A method of treating, preventing, or inhibiting acne or rosacea comprising administering to a subject in need thereof between 2% w/w to about 10% w/w benzoyl peroxide and from about 0.25% w/w to about 5% minocycline or a pharmaceutically acceptable salt thereof, wherein the benzoyl peroxide and the minocycline are co-administered by administration of a single combination composition, or by separate administration of (i) a first composition comprising benzoyl peroxide followed by administration of a second composition comprising minocycline or a pharmaceutically acceptable salt thereof; or (ii) a second composition comprising minocycline or a pharmaceutically acceptable salt thereof followed by administration of a first composition comprising benzoyl peroxide.

14. The method of claim 13, wherein the benzoyl peroxide and the minocycline is sequentially or concomitantly administered.

15. The method of claim 12, wherein the administration is by once daily or twice daily topical administration to a patient in need thereof.

16. The method of claim 15, wherein the once daily or twice daily administration is for up to 2 weeks, up to 1 month, preferably up to 2 months and more preferably up to 3 months.

17. The method of claim 12, wherein the benzoyl peroxide and the minocycline are formulated as the same dosage forms or different.

18. The method of claim 17, wherein the dosage forms are selected from a cream, a lotion, a gel, an ointment, an emulsion, a solution, a suspension, an elixir, a tincture, a paste, a foam, an aerosol or a spray.

19. The method of claim 17, wherein the benzoyl peroxide and the minocycline are both cream, lotion, ointment or a gel.

20. The method of claim 17, wherein the benzoyl peroxide dosage form is a cream and the minocycline dosage form is an ointment.

21. The method of claim 12, wherein the benzoyl peroxide is optionally encapsulated.

22. The method of claim 12, wherein the benzoyl peroxide is encapsulated, coated, adsorbed, embedded, impregnated, dispersed, entrapped, or encased in a polymeric material and providing a sustained release formulation.

23. The method of claim 12, wherein the minocycline is minocycline HCl.

24. A kit comprising a first composition comprising from about 2% w/w to about 10% w/w benzoyl peroxide, and a second composition comprising from about 0.5% w/w to about 10% w/w of minocycline or a pharmaceutically acceptable salt thereof, wherein the two compositions are mixed before applying on the skin of a patient in need thereof, thus administering a combination product; or administering the first and second composition sequentially.
